(19) United States
(12) Patent Application Publication
(10) Pub. No.: US 2022/0328143 A1
(43) Pub. Date: Oct. 13, 2022

(54) MACHINE LEARNING POST-TREATMENT SURVEY DATA ORGANIZING

(71) Applicant: Keely Kolmes, Oakland, CA (US)

(72) Inventor: Keely Kolmes, Oakland, CA (US)

(21) Appl. No.: 17/228,465

(22) Filed: Apr. 12, 2021

(51) Int. Cl. G16H 10/20; G06N 20/00; G16H 10/60

(52) U.S. Cl. CPC G16H 10/20; G06N 20/00; G16H 10/60

(57) ABSTRACT

Described herein are techniques, methods, systems, apparatus, and computer program products for processing post-treatment survey data for care providers. In certain embodiments, machine learning is utilized to provide a technique that removes identifying information from post-treatment surveys, in order to conform with professional ethics requirements. In another embodiment, a system for providing post-treatment surveys and determining feedback accordingly is provided.

### TECHNICAL FIELD

The present disclosure generally relates to analysis of post-treatment surveys. More specifically, the present disclosure relates to analysis of post-treatment surveys, such as post-treatment satisfaction surveys, that are provided for patients of psychotherapy providers and, specifically, to systems and machine learning techniques for conforming the results of such surveys with the professional ethics requirements for such psychotherapy providers.

### BACKGROUND

Professional ethics requirements for psychotherapy providers place stringent limitations on the use of patient testimonials or feedback. For example, psychotherapy or mental health providers, under the professional ethics requirements, are not allowed to solicit testimonials from ongoing patients (e.g., for marketing purposes) and patients where there might be undue influence.

### SUMMARY

Provided are techniques for processing post-treatment satisfaction data for care providers. In a certain embodiment, a method may be provided. The method may include receiving a training treatment survey, classifying portions of the training treatment survey as, at least, identifying sections or non-identifying sections, training, with the identifying sections, a first machine learning model to determine identifying data of surveys, training, with the non-identifying sections, a second machine learning model to determine an improvement point, receiving a post-treatment survey associated with a patient of a provider, determining, with the first machine learning model, one or more identifying data sections of the post-treatment survey, anonymizing the identifying data sections of the post-treatment survey to create an anonymized post-treatment survey, analyzing the anonymized post-treatment survey to determine an improvement point for the provider, and outputting the improvement point to a user device of the provider.

In another embodiment, a system may be provided. The system may include an electronic health record (EHR) database, a communications interface, communicatively coupled to the electronic health record (EHR) database via a network, a processor, communicatively coupled to the communications interface and the EHR database and configured to cause the system to perform operations. The operations may include receiving, with the communications interface, treatment process data from the EHR database, determining, from the treatment process data, that a treatment for a first patient with a first provider has finished, causing, based on the determining that the treatment for the first patient has finished, a post-treatment survey to be provided to an electronic device of the first patient, receiving a first survey, wherein the first survey is a response to the post-treatment survey, dividing the first survey into identifying sections and non-identifying sections, removing the identifying sections, determining, based on the non-identifying sections, a first improvement point for the first provider, determining, from course data stored within the EHR database and based on the first improvement point, a first course associated with the first improvement point, and transmitting first course.

These and other embodiments are described further below with reference to the figures.

### BRIEF DESCRIPTION OF THE DRAWINGS

The included drawings are for illustrative purposes and serve only to provide examples of possible structures and operations for the disclosed inventive systems, apparatus, methods and computer program products described herein. These drawings in no way limit any changes in form and detail that may be made by one skilled in the art without departing from the spirit and scope of the disclosed implementations.

FIG. 1 illustrates a representation of a system for determining post-treatment survey data, in accordance with one or more embodiments.

FIG. 2 illustrates another representation of a system for determining post-treatment survey data, in accordance with one or more embodiments.

FIG. 3 illustrates a flow process of a survey anonymizing machine learning training procedure, in accordance with one or more embodiments.

FIG. 4 illustrates a flow process of a survey anonymizing and review procedure, in accordance with one or more embodiments.

FIG. 5 illustrates a response analysis procedure, in accordance with one or more embodiments.

FIG. 6 illustrates a block diagram of a computing system, in accordance with one or more embodiments.

FIG. 7 illustrates a neural network, in accordance with one or more embodiments.

### DETAILED DESCRIPTION OF PARTICULAR EMBODIMENTS

This disclosure describes techniques, methods, systems, apparatus, and computer program products for processing post-treatment survey data (e.g., post-treatment satisfaction survey data) for psychotherapy or mental health providers (e.g., therapists) as well as other care providers or practitioners. As described herein, “practitioner” or “care provider” may refer to psychotherapy providers, but may also refer to other mental health providers, other therapists, and/or other service providers (e.g., medical doctors, attorneys, agents, and/or other such service providers), especially such practitioners or care providers that may be subject to codes of ethics or other duties. In certain embodiments, machine learning is utilized to provide a technique that determines identifying information and testimonials provided in post-treatment surveys. In order to conform with professional ethics requirements, such identifying information and testimonials are then removed from any published data (e.g., aggregate representations of data from the surveys). Such techniques utilize a specific configuration of machine learning systems in order to perform anonymizing and testimonial removal that is required by the professional ethics requirements.

Furthermore, in another embodiment, a system for providing post-treatment satisfaction surveys and determining feedback accordingly is provided. Such a system allows for post treatment satisfaction surveys to be provided according to professional ethics requirements, such as being provided only to patients that have finished treatment and are not under undue influence (e.g., the patient is not dependent upon the practitioner or care provider for any future service) from the practitioner or care provider. Furthermore, such a system configuration minimizes the interactions of care providers with patients, allows for greater convenience to patients, and allows for efficient processing of feedback due to the transformation of survey data into data usable for practice development.

Typical online review techniques gather testimonials independent of the clinical relationship as, and as such, do not provide for direct communication between the patient and the provider as to the patient treatment experience. Requesting an online review is also not in accordance with professional ethics requirements. The system and techniques described herein address such deficiencies. Furthermore, such online review techniques tend to result in survey respondents only posting reviews if they had an unusually positive or negative experience, or wish to provide a counterpoint to an already existing review, and no useful data is collected for the practice aspects (e.g., timeliness, booking techniques, billing techniques, mannerisms, and/or other such aspects) of the practitioner. The data collected may or may not provide information that consumers actually wish to obtain about various providers. The techniques described herein allow for collection of such practice data.

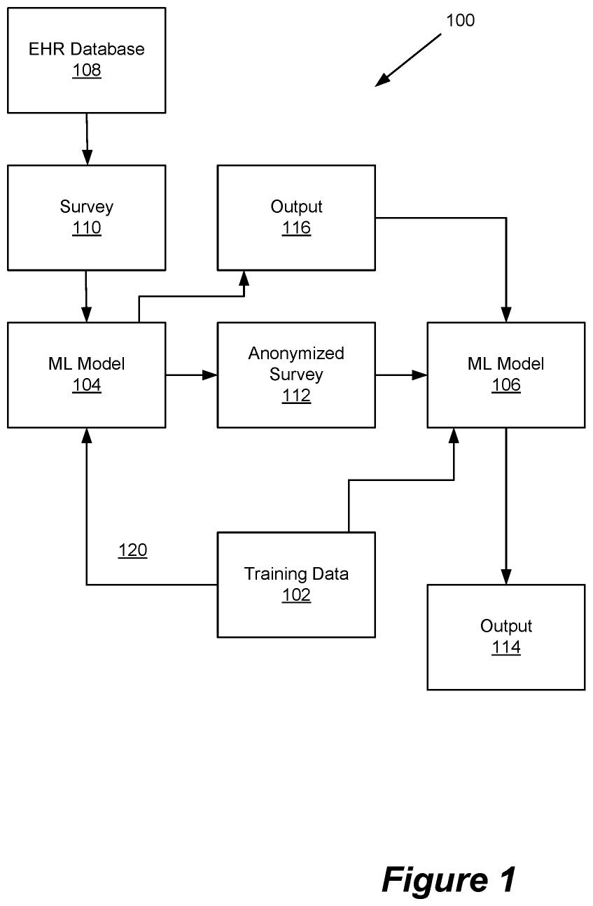

FIG. 1 illustrates a representation of a system for determining post-treatment survey data, in accordance with one or more embodiments. FIG. 1 illustrates system 100 that includes training data 102, machine learning models 104 and 106, and electronic health record (EHR) database 108. Various components of system 100 (e.g., machine learning models 104 and 106, survey 110, training data 102, and/or EHR database 108) may be configured to communicate data via communications channels 120.

Communications channels 120 may include any combination of wired (e.g., Ethernet, wired internet, phone lines, and/or other such techniques) and/or wireless (e.g., WiFi, 3G, 4G, 5G, Near Field Communications, and/or other such wireless techniques) communications channels and techniques for communicating data.

EHR database 108 may include data directed to one or more patients receiving treatment and/or one or more practitioners providing treatment. In certain embodiments, EHR database 108 may be configured to collect and/or store health data of various patients (e.g., patients receiving treatment that may respond to survey 110 upon finishing treatment). Data contained within EHR database 108 may include, for example, protected health information (PHI) such as name, date of birth, geographical identifiers such as addresses, city of residence, or other residence information, dates directly related to an individual, phone numbers, fax numbers, e-mail addresses, social security numbers, medical record numbers, health insurance beneficiary numbers, account numbers, certificate/license numbers, vehicle identifiers and serial numbers, device identifiers and serial numbers, Uniform Resource Locators (URLs), Internet Protocol (IP) addresses, biometric identifiers, images such as full face images, and/or any other unique identifying information, characteristic, or code, as well as other information not defined as PHI such as current treatment, treatment received, medical history, medication and allergies, immunization status, diagnosis, demographics information, personal statistics, and/or other such data. EHR database 108 may be configured to determine and/or provide data associated with a treatment status of a patient.

As such, EHR database 108 may be configured to determine when a patient has finished treatment and, based on such a determination and the requirements of professional ethics, provide survey 110 to the patient (e.g., via communication channels 120). In certain embodiments, the patient may provide an indication that the patient agrees to receive survey 110 after treatment has finished (e.g., in a confirmation message provided from an electronic device of the patient's or as part of patient intake forms and/or other informed consent documents). EHR database 108 may accordingly store data indicating that the patient has agreed to receive survey 110 after treatment for the patient has ended and conditions have been met (e.g., that no undue influence is determined). Certain examples of conditions that lead to a determination of no undue influence include, for example, the practitioner providing feedback to EHR database 108 (e.g., via a questionnaire such as a checkbox response) that there are no further plans for the patient to return, determining that there are no follow up appointments booked for the patient with the practitioner, providing for a pre-determined “cooling off period” (e.g., for a number of weeks such as 4-6 weeks) to allow for any changes in plan, indications within the survey or within responses by the practitioner and/or patient of expectations as to whether the patient would return, providing for one or more survey questions and then determining intent to return from answers provided (e.g., from the patient), and/or other such techniques. In various embodiments, machine learning may be utilized to determine the likely intentions or future actions of the patient, whether conscious or unconscious.

Thus, EHR database 108 allows for surveys to be provided to patients only after treatment has ended, allowing practitioners to receive feedback from patients in accordance with professional ethics rules. In certain embodiments, providing of survey 110 may be delayed until a threshold amount of time has passed where treatment has been determined to have ended, in order to prevent any situation where a survey is communicated before a patient has received additional follow-up treatment.

Survey 110, as described herein, may include one or more questions where a patient may provide feedback as to the practice of the practitioner or care provider. In certain embodiments, EHR database 108 may store one or more standard surveys and such standard surveys may be selected to be provided to the patient. However, in other embodiments, the practitioner or care provider may modify surveys provided to their patients by, for example, changing, deleting, and/or adding questions to the surveys. As such, the surveys may be customized for the practitioner. In certain embodiments, the techniques described herein (e.g., utilizing machine learning) allow for one or more different surveys to be utilized while being comparable for ranking/rating purposes.

In certain such embodiments, machine learning models 104 and/or 106 may be trained to standardize responses to surveys with different questions or formats. Thus, for example, machine learning models 104 and/or 106 may be trained to analyze the wording of the questions and determine whether the questions are leading or biasing responses from the respondent. Thus, for example, machine learning models 104 and/or 106 may analyze typical response biases to various question wordings. The machine learning models 104 and/or 106 may then provide a modifier to responses to the questions based on the wording or the questions themselves. As such, though the practitioner may include customized surveys, the ratings provided for the practitioner based on the survey responses, which are used for comparison of the performance of the practitioner to that of other practitioners, may be based on standardized responses so that the ratings are directly comparable and fair.

The patient may then provide feedback through survey 110. Survey 110 may then be filled out by the patient and provided to EHR database 108 and/or machine learning models 104 and/or 106 (e.g., via communication channels 120). Thus, in various embodiments, survey 110 may be received by machine learning models 104 and/or 106 or received by EHR database 108 and provided to machine learning models 104 and/or 106 by EHR database 108 for analysis and anonymizing, as described herein.

Survey 110 may include questions where a respondent (e.g., a patient taking the survey) may provide feedback through alphanumerical ratings (e.g., number ratings or letter grades). In various embodiments, survey 110 may be a post-treatment large scale satisfaction survey directed to various aspects of practice (e.g., not limited to effectiveness of the treatment). The responses to survey 110 may allow for a practitioner to improve their practice from a holistic perspective (e.g., from more than simply an effectiveness perspective).

Survey 110 may include multiple choice questions, questions allowing for a respondent to provide an alphanumerical response (e.g., letter grade or number rating), one or more selections for answers (e.g., a selection within a multiple choice question), and/or other such feedback. In certain embodiments of survey 110, the respondent may additionally provide textual feedback (e.g., in the form of sentences or paragraphs). In certain such embodiments, the textual feedback and/or other responses may include identifying information (e.g., provided by the user by accident and/or in response to questions of survey 110) or testimonials. Machine learning models 104 and/or 106 may be configured to determine such identifying information or testimonials, remove, anonymize, and/or separate such information, and/or analyze the responses to survey 110 to provide feedback and ratings for the practitioner, in accordance with the professional ethics requirements.

Machine learning models 104 and 106 may be various machine learning models configured to be trained with training data 102. Machine learning models 104 and/or 106 may be trained to receive data and manipulate such data accordingly. Accordingly, machine learning models 104 and/or 106 may be configured to receive data as inputs and provide one or more outputs based on the inputs. In certain embodiments, machine learning models 104 and/or 106 may be configured with one or more neural networks or other learning models.

Machine learning models 104 and/or 106 may include text recognition configured to understand textual responses provided by the respondents within the surveys. Textual recognition allows for machine learning models 104 and/or 106 to analyze survey responses (e.g., textual responses) from respondents and manipulate and/or determine data from such survey responses according to the techniques described herein.

Machine learning models 104 and/or 106 may additionally be configured to receive data from EHR database 108. Such data may be, for example, data associated with the identity of a respondent (e.g., name, date of birth, address, city of residence, treatment received, and/or other such data such as PHI data). Such data may be received by machine learning models 104 and/or 106 (e.g., communicated from EHR database 108 and/or requested by machine learning models 104 and/or 106) and utilized to determine identifying data, essay data (e.g., written descriptions), and/or testimonials within survey responses.

Machine learning model 104 may be trained by training data 102 to receive survey data (e.g., from survey 110) and determine, at least, identifying and/or non-identifying data as well as testimonials within the survey data. In various embodiments, identifying data may be any data that allows for determination of an identity of a respondent to the survey or PHI. Thus, for example, names, date of birth, addresses, city of residence, gender, orientation, personal preferences, types of treatment received, progress of treatment, results of treatment, and/or other such data such as other PHI data may be determined by machine learning model 104. Machine learning model 104 may be configured to remove and/or modify such identifying data to anonymize the survey data.

Accordingly, for example, the identifying data may be deleted and/or modified to avoid identifying references. As such, for example, the name of the respondent that is identified within the survey may be deleted and/or changed to a placeholder name (e.g., Jane Doe). In another example, the date of birth or address may deleted or replaced. In a further example, the survey may mention a type of treatment received. As the sentence and/or paragraph containing mention of the type of treatment received may include other identifying data (e.g., the reason for the treatment and/or the results), machine learning model 104 may be trained to completely remove the sentence and/or paragraph containing the type of treatment received and/or further analyze the sentence and/or paragraph to identify the other identifying data and anonymize the other identifying data accordingly.

Alternatively or additionally, certain embodiments may be configured to remove or keep mentions of types of treatment. Thus, for example, in certain situations, the type of treatment may be non-identifying and may provide useful information as to the performance of a practitioner in aggregate. Nonetheless, in certain other situations, type of treatment may be determined to be identifying (e.g., when the description for treatment received includes the names of the practitioner and/or which sessions certain situations happened within) and such identifying information may be removed. Furthermore, in certain embodiments, though the type of treatment may be utilized in determining feedback, rankings, and/or ratings, such individual information may not be presented to viewers of the data (e.g., one or more of practitioners and/or members of the public accessing the data).

Machine learning model 104 may accordingly modify survey 110 and/or generate anonymized survey 112 based on survey 110. Anonymized survey 112 may be survey 110 with identifying data related to the identity of the respondent removed and/or modified (e.g., anonymized as described herein). Anonymized survey 112 may then be provided to machine learning model 106.

Certain responses may be identified as essays. Essays may be, for example, written responses of the respondent. Essays may include testimonials. Testimonials may be any written or verbal description of the practice of the practitioner or care provider. In certain situations, testimonials may include an endorsement of the practitioner or care provider. In other situations, testimonials may include identifying data of the respondent and/or of other parties, descriptions of treatment discussed and/or received, impressions from the respondent, and/or other such descriptions. In certain embodiments, any textual response may be considered to be testimonial in nature.

Testimonials may be analyzed (e.g., for preparing ratings or outputs), removed (e.g., to provide for textual feedback to the user), deleted, and/or otherwise manipulated. In certain embodiments, machine learning model 104 may be configured to identify portions of survey 110 that are testimonial or potentially testimonial (e.g., if the sections include textual responses and/or include responses that machine learning model 104 has learned as potentially testimonial). Machine learning model 104 may then accordingly remove, delete, or otherwise flag these sections. In certain embodiments, where machine learning model 104 is configured to remove the testimonial sections, such testimonial sections may be provided as output 116. Output 116 may be an output such as a summary or testimonial data that is provided to the practitioner (e.g., an electronic device of the practitioner). In certain embodiments, output 116 may include the text of the testimonial data. In certain fields, though professional ethic rules prevent practitioners from publishing testimonials (e.g., for marketing or advertising purposes), testimonials may still be useful for the practitioner to improve aspects of their practice. Accordingly, output 116 may be provided as a separate output to allow for further improvement.

In certain such embodiments, output 116 and output 114 may be output together. Additionally or alternatively, publication of reviews, data, ratings, and/or other data may be performed by EHR database 108 or the entity associated with EHR database 108. Such publication may be of output 114 that does not include testimonials. Nonetheless, output 116, including the testimonials, may be provided to the practitioner for effective feedback, but not for publication, which may remove identifying data and/or testimonials, to conform with ethics rules.

Machine learning model 106 may be trained by training data 102 to analyze anonymized survey 112 and provide output 114 according to the analysis. In a certain embodiment, machine learning model 106 may determine one or more such outputs based on anonymized survey 112. That is, machine learning model 106 may be configured to receive survey 112, as well as other surveys, that has been anonymized by machine learning model 104, and make such determinations based on anonymized survey 112. Thus, for example, based on analysis of anonymized survey 112, machine learning model 106 may determine one or more aspects of practice that the care provider may improve on. Machine learning model 106 may make such a determination based on analysis of alphanumerical feedback and/or textual feedback (e.g., through textual analysis).

Machine learning model 106 may, thus, be trained to determine, based on the responses within anonymized survey 112, ratings within one or more practice categories (e.g., empathy, billing, communications, humor, and/or other such categories) for the practitioner. Furthermore, machine learning model 106 may be configured to determine (e.g., based on the ratings) one or more practice categories where the practitioner is weak or strong (e.g., empathy, billing, communications, humor, and/or other such categories). Such ratings and/or categories may be provided as output 114. Such ratings, as all testimonial data and identifying data may be removed from such ratings, may be suitable for publication by the practitioner or care provider (e.g., for marketing or comparison purposes).

In certain embodiments, surveys may ask patients if they understand their diagnosis. Machine learning model 106 may adjust, based on the response provided by the patient, the ratings/rankings determined from anonymized survey 112 based on the response given by the respondent. Thus, for example, a survey where the respondent does not understand their diagnosis may be weighted in a different manner (e.g., upweighted or downweighted), for determining rankings, than surveys where the respondent understands their diagnosis.

Additionally, output 114 may include further determinations based on the ratings and/or categories. Such outputs may be, for example, a suggested class, resource, tutorial, and/or other output that may allow the care provider to improve in the determined area or continue to perform well in such areas. Output 114 may include data that allows for the care provider to access or utilize such resources, such as contact information, a Uniform Resource Locator (URL) link, an e-mail address, a way to sign up, a meeting request, and/or other such data.

In certain embodiments, the suggested class, resource, tutorial, and/or other output may be stored within EHR database 108. As such, machine learning model 106 may determine the one or more practice categories and then receive data from EHR database 108 (e.g., in response to a request from machine learning model 106) to identify the one or more of the suggested class, resource, tutorial, and/or other output. For example, based on the determination of the categories (e.g., one of the categories may be that of billing), machine learning model 106 may then provide a request to EHR database 108 for all possible resources that allow a care provider to improve their performance in that category (e.g., classes on billing, additional billing service providers, reminder services, and/or other such resources). EHR database 108 may then provide data indicating such possible resources. Such resources may be provided within output 114.

Machine learning model 106 may be additionally trained to select appropriate resources from that provided by or stored within EHR database 108. For example, machine learning model 106, in a first example, may determine that a practitioner and/or care provider requires improvement in billing services. Additionally, based on analysis of anonymized survey 112, machine learning model 106 may determine that the respondent indicated, through a textual response, that the care provider included billing policies that were felt to be too aggressive. In certain embodiments, such a determination may be made, additionally or alternatively, from output 116. In such an embodiment, output 116 may include testimonial data that is anonymized, in order to prevent divulging of the identity of the respondent. Machine learning model 106 may then determine, from data associated with the care provider stored within EHR database 108, that respondents are dissatisfied with the care provider's cancellation policies. Machine learning model 106 may accordingly select business practice training resources stored within EHR database 108 and provide data related to such business practice training resources (e.g., contact or sign-up information) as output 114 to the practitioner and/or care provider, so that the practitioner and/or care provider may consider adjustments to their cancellation policies. The configuration of system 100 may allow for points of practice improvement to be determined for a care provider while maintaining the privacy of patients and/or respondents (e.g., the patients providing feedback) and for resources to be provided to the care provider to help improve in such areas.

Training data 102 may be used to train machine learning models 104 and/or 106. Training data 102 may be previously provided surveys and/or mock-up surveys configured specifically to be used as training data. Training data 102 may include one or more alphanumerical responses and/or textual responses. Training data 102 may, in certain portions thereof, include identifying data of respondents.

In certain embodiments, training data 102 may be utilized to simultaneously train machine learning models 104 and 106. In other embodiments, training data 102 may be utilized to train machine learning models 104 and 106 at different times. However, it will be appreciated that, in certain such embodiments, the same training data 102 is utilized to train both machine learning models 104 and 106. Thus, though machine learning model 106 in operation receives anonymized data (e.g., machine learning model 106 receives anonymized survey 112 that has been anonymized by machine learning model 104), machine learning model 106 is trained with training data that still includes identifying information. As, in operation, machine learning model 104 may not perfectly anonymize survey 110, training machine learning model 106 with training data 102 that includes identifying data may allow machine learning model 106 to determine improvement categories without disclosing identifying information, allowing for system 100 to fully conform with professional ethics obligations. Furthermore, though machine learning models 104 and 106 receive different data in operation (e.g., non-anonymized data for machine learning model 104 and anonymized data for machine learning model 106), training both machine learning models 104 and 106 with the same training data 102 results in more resource efficient training of machine learning models 104 and 106, in the aggregate.

In certain embodiments, machine learning models 104 and/or 106 may be further trained in operation. Thus, survey 110 and/or anonymized survey 112 may be further used to train machine learning models 104 and/or 106. In certain embodiments, any remaining identifying data or testimonial remaining within anonymized survey 112 (or survey 110) may be further identified and such identification may be further used to train machine learning models 104 and/or 106. As well, data for determining the ratings and/or output may additionally be identified and used to train machine learning models 104 and/or 106.

In a certain example, the definition of identifying data may change over time. For example, new contact techniques or platforms (e.g., social media platforms) may lead to additional forms of contact data. Machine learning models 104 and/or 106 may be trained to identify new identifying data as they develop. Thus, for example, machine learning models 104 and/or 106 may identify new types of identifying data that respondents provide (e.g., during information portions of the surveys) and be trained to determine that further instances of such data are identifying data.

In another example, what is considered to be testimonial in nature by the standards of professional ethics may change over time. Machine learning models 104 and/or 106 may be configured to adapt to such changes in the standards of professional ethics. For example, additional training data may be provided to machine learning models 104 and/or 106 when professional ethics standards change. In another example, practitioners or other proof readers may flag any testimonial data remaining within output 114. Such flagged testimonial data may be provided to further train machine learning models 104 and/or 106 to identify testimonial data (e.g., based on changes within the standards of professional ethics).

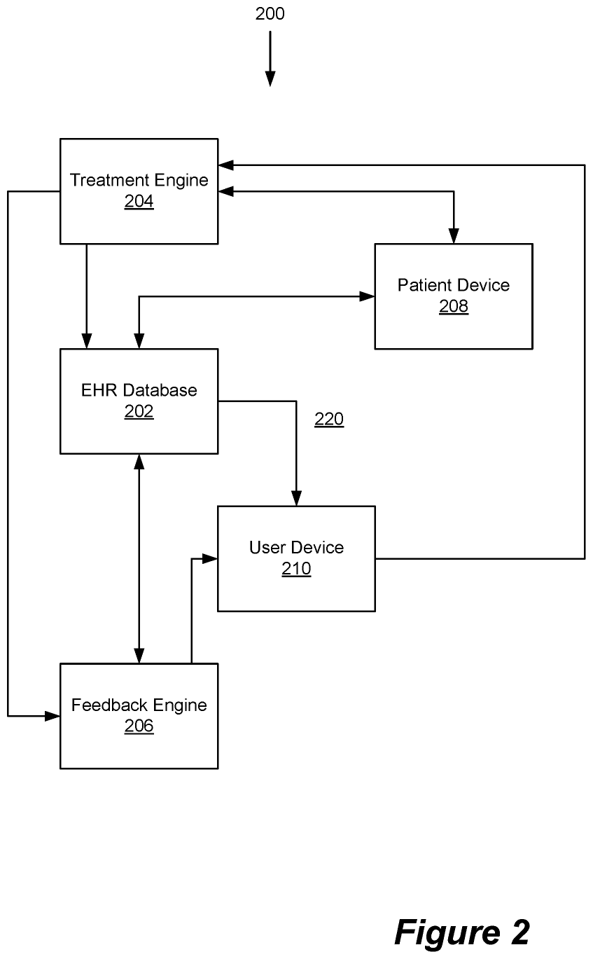

FIG. 2 illustrates another representation of a system for determining post-treatment survey data, in accordance with one or more embodiments. FIG. 2 illustrates system 200 that includes EHR database 202, treatment engine 204, feedback engine 206, patient device 208, care provider user device 210, and communications channel 220. In various embodiments, patient device 208 may be one or more electronic devices (e.g., desktop computer, laptop computer, server device, smart phone, tablet, wearable device, and/or another such electronic device) associated with a patient and/or survey respondent and utilized by the patent and/or survey respondent. EHR database 202, treatment engine 204, feedback engine 206, patient device 208, and/or care provider user device 210 may communicate, directly or indirectly via communications channel 220. The various portions of system 200 (e.g., EHR database 202, treatment engine 204, feedback engine 206, patient device 208, and/or user device 210) may each include one or more communications interfaces configured to communicate via communications channel 220.

EHR database 202 may be similar to EHR database 108 described in FIG. 1. That is, EHR database 202 may be configured to store patient, population (e.g., patient population), and/or care provider information, as described herein. Furthermore, EHR database 202 may be configured to store data associated with classes, tutorials, and/or other resources that allow for a care provider to improve the care provider's practice. EHR database 202 may be one or more databases that are stored within one or more server devices (e.g., within hard drives or other memory storage devices) and/or within cloud databases.

Treatment engine 204 may be configured to track the status and/or progress of patient device 208's treatment. Thus, for example, treatment engine 204 may receive data from EHR database 202 indicating upcoming appointments of patient device 208. Treatment engine 204 may accordingly provide reminders to patient device 208 of the upcoming appointments. Furthermore, treatment engine 204 may receive data from patient device 208 regarding whether treatment has been completed. Treatment engine 204 may receive such data and generate data appropriate for EHR database 202 (e.g., in a format that EHR database 202 may utilize to update the status of patient device 208 stored within EHR database 202).

Accordingly, treatment engine 204 may indicate to EHR database 202 when a patient has finished treatment. In certain embodiments, before the beginning of treatment, EHR database 202 and/or treatment engine 204 may provide an indication as to whether the patient agrees to receive the survey after treatment and such data may be accordingly stored within EHR database 202. EHR database 202 and/or treatment engine 204 may, after determining that treatment has ended for a patient, confirm whether the patient has agreed to receive a post-treatment satisfaction survey, from data stored within EHR database 202. Based on the indication, EHR database 202 may communicate a survey to patient device 208. Patient device 208 may include a user interface that may receive inputs from the patient to complete the survey and may then provide a completed survey to either EHR database 202 and/or treatment engine 204 (e.g., depending on the configuration of system 200). EHR database 202 and/or treatment engine 204 may then provide the survey to feedback engine 206.

In certain embodiments, treatment engine 204 may be configured to determine when treatment has ended. Thus, for example, a practitioner, with user device 210, may indicate if a patient's treatment is active or inactive. If the patient's treatment is indicated to be inactive, treatment engine 204 may determine that a patient's treatment has ended and, thus, a survey may be communicated to patient device 208. In other embodiments, a patient's treatment may be indicated to end on a certain date or that the patient's treatment includes a set number of visits to the practitioner. In such embodiments, treatment engine 204 may determine that the ending date has passed or that the patient has gone through the set number of visits. Treatment may accordingly be determined to have ended and the survey provided.

Feedback engine 206 may be configured to determine feedback for practitioners as well as determine resources associated with the feedback. Feedback engine 206 may be configured to receive one or more surveys from patient device 208 (e.g., directly via communications channel 220), treatment engine 204, and/or EHR database 202. Feedback engine 206 may include one or more machine learning models, such as machine learning models 104 and/or 106 described in FIG. 1. As such, feedback engine 206 may receive the survey, anonymize the survey, remove and/or provide testimonial data, determine one or more improvement points, and/or determine one or more resources (e.g., from EHR database 202) for the practitioner based on the improvement points.

In various embodiments, feedback engine 206 may receive data from treatment engine 204 and/or EHR database 202 during operation of feedback engine 206 (e.g., performing the techniques described herein). Such data may be for indicating the status of the patient (e.g., the patient associated with patient device 208). Such data may indicate, for example, whether the patient is still in treatment or has finished treatment. Such data may allow feedback engine 206, after receiving the survey, to confirm whether the patient has finished treatment. For example, certain patients may, after finishing treatment and being provided with a survey, restart treatment for whatever reasons. EHR database 202 may, in such examples, receive data indicating that the patient has restarted treatment. Surveys may include identifying data (e.g., surveys may ask for the patient to provide their name, date of birthday, insurance number, PHI data, and/or other such data) and such identifying data may be used (e.g., by machine learning model 104) to match the survey with the identity of a patient that has data communicated and/or stored within treatment engine 204 and/or EHR database 202. If the data indicates that the patient has finished treatment, feedback engine 206 may then anonymize and analyze the survey, in accordance with the techniques described herein. If the data indicates that the patient has not finished treatment, feedback engine 206 may provide feedback to EHR database 202 and/or treatment engine 204 indicating that the survey was provided prematurely. Furthermore, such data may be used during the determination of various ratings and/or outputs. In certain embodiments, (e.g., after determining that the patient has finished treatment) EHR database 202 and/or treatment engine 204 may further determine if the patient has consented to receiving a survey after treatment, as described herein.

Such configurations of system 100 and/or 200 allow for data to be accessed from EHR database 202 accordingly to the characteristics of the survey, without requiring data to be continuously populated within feedback engine 206, allowing for more efficient usage of memory within the machine learning models of feedback engine 206 and improved processing, as only data that may be necessary for determining whether the patient should have been provided with the survey may be accessed from EHR database 202. Additionally, the configuration of system 100 and/or 200 allows for EHR database to provide data to feedback engine 206 and, accordingly, control the weighting and/or determination of various practice ratings. Thus, the configurations of system 100 and/or 200 allow for EHR databases to control the operation of machine learning models without directly modifying the machine learning models. As such, the systems described herein may be adapted to various EHR databases without the costly and timely need to reconfigure such machine learning models.

As such, in certain embodiments, feedback engine 206 and/or the machine learning models may be configured to interface with a plurality of different EHR databases. In such an embodiment, feedback engine 206 and/or one or more of the machine learning models may be configured to determine the specific EHR database that it is receiving data from by, for example, the format of the data, the various data provided, and/or through another characteristics. Such a configuration allows for feedback engine 206 to provide service to a plurality of different service providers without requiring different feedback engines and/or machine learning models for each service provider.

User device 210 may be an electronic device associated with a care provider. Thus, user device 210 may be an electronic device such as a desktop computer, laptop computer, server device, smart phone, tablet, wearable device, and/or another such electronic device, and/or another such device. User device 210 may be configured to communicate via communications channel 220 with the rest of system 200 and may, for example, receive one or more outputs from feedback engine 206. Such outputs may be, for example, one or more improvement points and/or resources associated with the improvement points. User device 210 may include a user interface such as a visual screen or audio output. The user interface may be configured to communicate (e.g., display or provide audio of) the output to the user (e.g., practitioner) of user device 210.

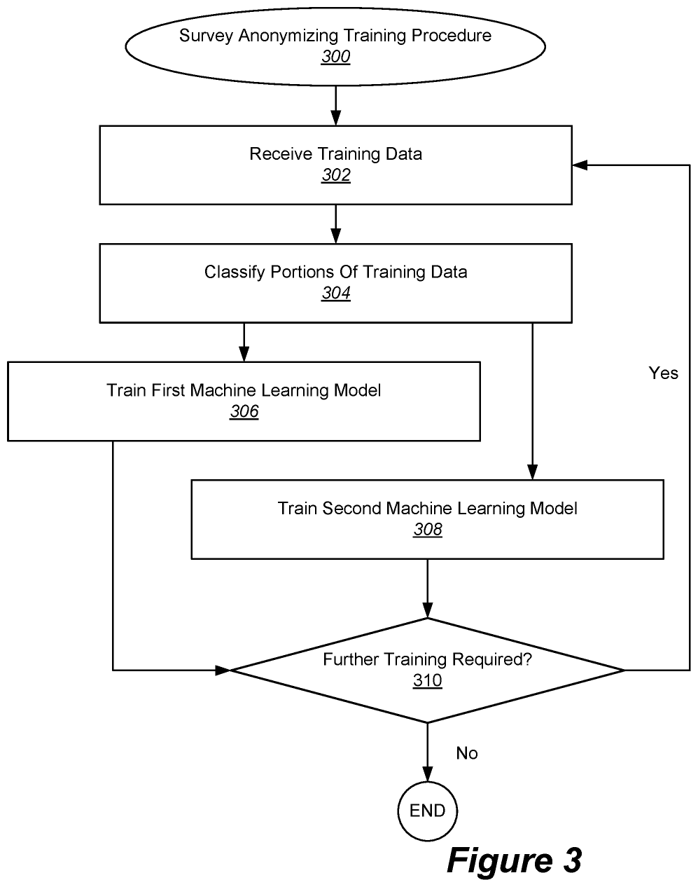

FIG. 3 illustrates a flow process of a survey anonymizing machine learning training procedure, in accordance with one or more embodiments. FIG. 3 illustrates survey anonymizing training process 300 that may allow for training of one or more machine learning models described herein.

In 302, training data is received by the machine learning model. Such training data may be surveys, either filled out by actual users or generated to serve as training data. The training data may include one or more alphanumerical responses and/or textual responses, as well as identifying data.

After receiving the training data, portions of the training data may be classified in 304. Classification of the training data may include, for example, classifying portions of the surveys of the training data as one or more of identifying data, feedback data (e.g., directed to one or more practice categories such as empathy, billing, timeliness, and/or other categories), testimonial data, numerical feedback, non-numerical feedback (e.g., commentary), non-useful data, and/or other types of data. Classification of training data may be performed by an electronic device, or manually.

The classified training data may then train the first machine learning model in 306 and the second machine learning model in 308. The machine learning model in 306 may be trained to remove or anonymize identifying data within surveys and/or determine testimonial data and remove, delete, and/or change testimonial data, as described herein. Thus, the machine learning model in 306 may be trained to analyze surveys to determine identifying data and modify and/or remove such identifying data as well as delete or remove testimonial data, while preserving them for feedback to be provided to the practitioner.

The machine learning model in 308 may be trained to determine one or more improvement categories and/or resources, as described herein. Thus, for example, the machine learning model in 308 may be trained to analyze surveys and determine categories that a practitioner can improve in.

In certain embodiments the machine learning model in 308 may be trained to withhold text completely (e.g., the feedback may be purely in category and alphanumerical ratings). Instead, the machine learning model in 308 may be trained to publish a dataset associated with the practice of the practitioner. In certain additional embodiments, the machine learning model in 308 may be configured to aggregate data. That is, the machine learning model in 308 may analyze a plurality of surveys (e.g., a number of surveys equal to or greater than a minimum number of surveys) and provide ratings and/or improvement points based on analyzing the data from the plurality of surveys. Such improvement point outputs may thus include the categories as well as a score or output indicating areas of improvement.

In certain embodiments, the machine learning model in 308 may utilize additional data from an EHR database or treatment engine. Accordingly, for example, the machine learning model in 308 may determine the location of one or more patients and/or the location of the practitioner. The improvement categories or other determinations of the machine learning model in 308 may be based on such locations. For example, potential patients may wish to compare practitioners in certain locations (e.g., located within certain ZIP codes) and/or for various aspects of service (e.g., empathy, accommodation of gender, cultural sensitivity, and/or other such aspects) and/or practitioners may wish to compare themselves to other practitioners within the same locations or for certain practices. The determination of improvement areas may be accordingly modified (e.g., the importance of a category may be adjusted based on trends within the certain locations, the determined proficiency of a practitioner may be adjusted upward if local practitioners within the area all score low, and/or another adjustment made, while the priority for improvement in such areas may also be adjusted if local patients all indicate that they value certain specific characteristics) based on the local area. Furthermore, survey data may, in some or all surveys, include location data directed to the location of the respondent. The machine learning model in 308 may accordingly be trained to analyze the location data and adjust the determination according to the location data of the survey.

In certain additional embodiments, machine learning model 308 may be configured to provide ratings and/or recommendations based on the specific practice category of characteristic of treatment or of the practitioner. That is, for example, machine learning model 308 may be trained to determine the category of care received by the patient and/or provided by the practitioner. Machine learning model 308 may then access data from the EHR database directed to the performance of other practitioners that are specifically within the category of care, practice field, geographical area, and/or other characteristics (e.g., expert in gender issues, providing counseling to bisexuals, accepting new patients) that matches the practitioner. The rating and output may then accordingly be determined from data of such categories that are received from the EHR database. Thus, comparable ratings and/or more appropriate outputs may be accordingly determined by machine learning model 308.

In further embodiments, the machine learning model in 308 may be trained to adjust for edge case responses. That is, survey responders may often be extremely satisfied or unsatisfied with the treatment provided while patients without a strong feeling for the services provided may form a larger portion of non-responders. The machine learning model in 308 may be trained to identify such edge case responding surveys (e.g., trained to identify that the responses are consistently extremely negative or consistently extremely positive) and decrease the importance of such surveys in determining the improvement points (e.g., the weight of such responses may be decreased). Accordingly, the machine learning model in 308 may be trained to identify patterns within survey responses that are indicative of such edge case responses.

As described herein, the same classified training data (e.g., classified in 304) may be used to train both the first machine learning model and the second machine learning model, so that second machine learning model may determine improvement categories without disclosing identifying information that may not have been removed.

In 310, a determination may be made whether further training of one or more machine learning models may be needed. If no further training is needed, training of the machine learning models may end. If further training is needed, the technique may return to 302 and receive further training data.

In certain embodiments, a determination that further training is needed may include, for example, a determination that professional ethics standards have changed (e.g., EHR database or another database may include professional ethics standards and any change within the standards may trigger further training), that additional training data is available, that a threshold amount of training data has not yet been provided to the machine learning models, that one or more regions or other categories require further training, that outputs from the machine learning models have been flagged for further training (e.g., indicating that identifying data still remain within the outputs), and/or another such determination. If such a determination is made, additional training of the machine learning model may be conducted.

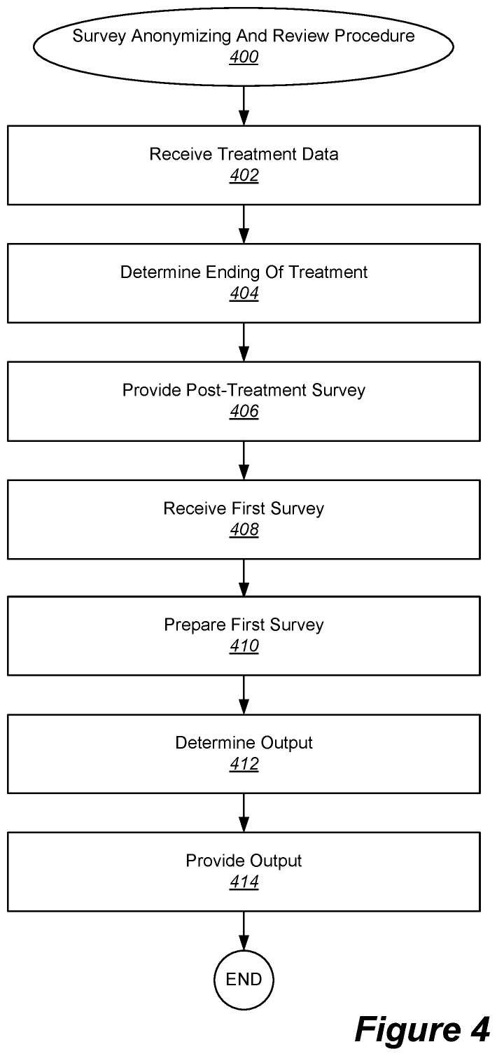

FIG. 4 illustrates a flow process of a survey anonymizing and review procedure, in accordance with one or more embodiments. FIG. 4 illustrates survey anonymizing and review procedure 400 that may allow for one or more machine learning models described herein to determine one or more ratings, improvement points, and/or other outputs. Various portions of survey anonymizing and review procedure 400 may be performed by portions of systems described herein, such as EHR database, treatment engine, feedback engine, and various machine learning models described herein.

In 402, treatment data may be received (e.g., from an EHR database, treatment engine, or an electronic device of a patient). The treatment data may be data associated with treatment of the patient, such as the stage of treatment of such treatment. The treatment data may be analyzed in 404 and, based on the analysis, a determination made that the patient has finished treatment. Furthermore, in certain embodiments, a patient may need to opt in to receiving a post-treatment survey. Treatment data may additionally indicate whether the patient has opted into the survey and only such patients may be provided the survey in 406.

Additionally or alternatively, embodiments may include a determination by the practitioner as to whether the patient is in a condition to receive the survey. For example, in certain situations, the practitioner may determine that receiving a survey, regardless of whether the patient had previously opted in, may be distressing to the patient. When the end of treatment is determined, the practitioner may be consulted (e.g., via an electronic questionnaire) to determine whether the patient is in a condition to receive the survey. In certain embodiments, the percentage of patients that a practitioner indicates as being unable to receive a survey may also be output, in order to prevent practitioners from only allowing patients with positive impressions to receive the survey. Also, certain embodiments may receive confirmation (e.g., via an electronic questionnaire) from a patient that they are willing to receive the survey before a survey is provided.

After providing the survey, the system may then receive a first survey in 408, which may be a response to a post-treatment survey provided to a patient. The first survey may include one or more responses or feedback to the post-treatment survey. The first survey may then be provided to the machine learning models, as described herein.

A first machine learning model may receive the first survey and prepare the first survey in 410. Preparation of the first survey may include, for example, anonymizing the first survey to remove identifying data within the first survey as well as identifying one or more categories for comparison to the survey (e.g., presenting problem, field of practice, geographical area, specific treatments, request for cultural competence, and/or other categories). The first survey may accordingly be anonymized and prepared for analysis (e.g., based on the categories).

After anonymizing of the first survey, the first survey may be analyzed and an output determined in 412. Analysis of the first survey may include, for example, determining one or more ratings within various categories of practice and/or one or more improvement points based on the feedback within the first survey. Accordingly, the second machine learning model may analyze the anonymized first survey and determine the ratings and/or improvement points accordingly. In certain embodiments, such ratings and/or improvement points may specific to the categories identified. Based on the improvement points, one or more outputs (e.g., techniques for improving based on the improvement points) may be determined (e.g., from data within EHR database and/or through other techniques, as described herein) in 414, according to the techniques described herein. The output may be provided (e.g., communicated to a user device) of the practitioner in 414.

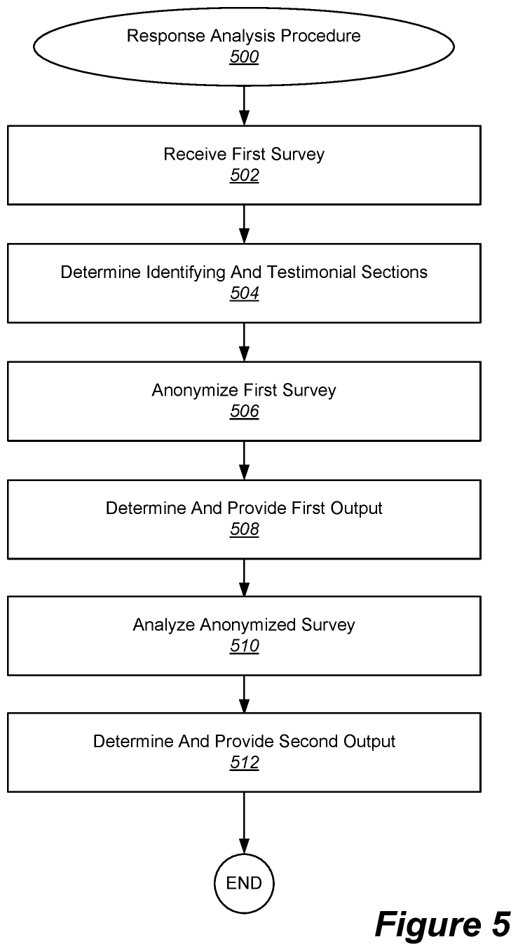

FIG. 5 illustrates a response analysis procedure, in accordance with one or more embodiments. FIG. 5 illustrates response analysis procedure 500. Response analysis procedure 500 may be a technique for analyzing survey responses (e.g., anonymized survey responses) to determine one or more improvement points for a practitioner.

In 502, a first survey is received. The first survey may be, for example, a completed post-treatment survey provided to a patient after a determination that treatment has ended. The first survey may be filled out and received from the patient.

After receiving the first survey, various portions of the first survey may be determined and categorized in 504. Thus, for example, portions of the first survey may be determined to be, at least, one of an identifying or non-identifying section or include identifying or non-identifying data. Additionally or alternatively, in other embodiments, portions of the first survey may be determined to include practice data (e.g., data indicating the performance of the practitioner in various categories), anecdotal data (e.g., stories provided by the respondent, which may or may not be identifying sections), administrative data, and/or other such data. In various embodiments, practice data may be additionally categorized to different practice fields (e.g., empathy, billing, humor, cultural sensitivity, and/or other such categories). The various different categories may be accordingly analyzed to determine practice ratings and/or improvement points (e.g., in 508).

After categorizing of the first survey, the first survey may be anonymized in 506. In certain embodiments, respondents may indicate (e.g., within the survey) whether they may wish for identifying data to be provided to the practitioner. In certain such embodiments, the anonymizing may be based on the indications of the respondent. The respondent may additionally indicate which portions of identifying information should be anonymized and the anonymizing of 506 may be performed accordingly. Such a configuration may increase respondent confidence that their privacy may be protected.

In certain embodiments, the surveys may include an option (e.g., checkbox) for the respondent to request that the practitioner contact the respondent to discuss their experience. Such options may result in an unpublished output to the practitioner indicating that the respondent wishes to be contacted to discuss. Other options may also be presented to the respondent within the survey, such as an option to be anonymized (e.g., the machine learning model may, upon determining that such an option has been selected, operate machine learning model in a configuration that further anonymizes the survey and/or deletes all identifying information from outputs provided to the practitioner instead of only deleting identifying information from published information) and/or other questions based on the responses provided by the respondent (such as the manner to be contacted if the option to be contacted is selected).

As such, for example, portions of the first survey that are categorized as identifying sections or include identifying data may be removed and/or changed so that the identifying information is changed to generic information or deleted, according to the techniques described herein. In various embodiments, 504 and 506 may be performed with a first machine learning model. Anonymizing of the first survey in 506 may result in an anonymized first survey that may be analyzed in 508.

In certain embodiments, the first survey may include testimonial data and such testimonial data may also be identified in 504. In 508, the testimonial data may be removed and/or deleted from the first survey. In certain embodiments, the testimonial data may be provided as a first output. The first output may allow for the practitioner to review written feedback for their practice. Under professional ethics rules, such feedback generally should not be published. Thus, first output may be an output that is incompatible with publication to, for example, a profile of the practitioner within the DAR database. As such, the first output may be a private output.

In 510, the anonymized first survey is analyzed by, for example, the second machine learning model. Analysis of the anonymized first survey may determine one or more practice ratings and/or improvement points, according to the techniques described herein. Thus, the performance of the practitioner in various categories may be determined by the second machine learning model. The performance may be provided in the form of an alphanumerical rating, textual description (e.g., describing the good and/or bad points of the practitioner's performance), and/or other description to provide information as to the practitioner's performance. Such ratings may be that of the practitioner's performance to a pool of other practitioners, such as practitioners within the same practice category or when compared to other practitioners providing the same type of treatment.

In a certain example, one or more responses to the survey may be converted to a numerical rating. In examples where a plurality of surveys are utilized to determine the practice ratings, one or more surveys may include a weight that may factor into the numerical rating (e.g., a highly weighted survey may figure more prominently within the numerical rating while a lower weight survey may not figure as prominently). Thus, the numerical rating for each category or aspect of the practitioner's practice may be weighted and/or modified by the weights. The numerical rating may provide for a score for the practitioner's practice and/or an aspect of the practitioner. The scores from each of the plurality of surveys may be weighted and a corresponding rating (e.g., total score, weighted average, median, and/or other way of determining the rating) may be determined. Such a rating may be the rating for the overall performance of the practitioner. In embodiments where a plurality of aspects of practice are rated, each aspect may include a separate rating.

In certain such embodiments, weighting of each question and/or aspect of practice of the survey may be adjusted based on data associated with the patient (e.g., the results of the treatment of the patient) and/or the practitioner. Such data may be accessed from the EHR database and/or treatment engine. Thus, for example, in a survey where the respondent was a patient that the EHR database indicates collection of payment from was troublesome (e.g., payment by the credit card of the patient was rejected multiple times or their bill was sent to collections), portions of the survey associated with the billing practices of the practitioner may be downweighted as the patient may be determined to have a high likelihood of annoyance with billing related matters. However, in such an example, portions of the survey directed to cultural sensitivity may not be downweighted as the practitioner may include a history of cultural insensitivity, based on data from the EHR database.

In certain embodiments, the pool of other comparable practitioners (e.g., offering the same treatment, or whose ratings are determined from surveys where the patients were receiving the same type of treatment) may be determined from the EHR database. Furthermore, in certain embodiments, the pool may be further sorted by location or geographical data, ethnicity data, and/or other such data that may be present within the EHR database. Thus, while the second machine learning model may utilize data from the EHR database to determine the performance of the practitioner, such a pool may be narrowed and/or changed based on the characteristics of the practitioner, the practice field of the practitioner, the locations, the patients, and/or other such aspects of practice.

Based on the practice ratings and/or improvement points, an output may be determined in 510. The output may include the ratings, one or more of practice advice (e.g., practice pointers, determined by the second machine learning model), one or more resources available to the practitioner (e.g., for improving the practitioner's practice), one or more classes available for improvement (e.g., stored within the EHR database), one or more services available to the practitioner (e.g., offered by the EHR database or a service provider associated with the EHR database), and/or another such resource. The output may be provided to practitioner's user device, in accordance with the techniques described herein.

Furthermore, the output may include rankings and/or ratings. Such rankings and/or ratings may include a numerical output and/or a ranking of the practitioner relative to the practitioner's peers (e.g., practitioners within the same field and/or geographical area, based on identifiers within the EHR database). Such rankings and/or ratings may include one or more categories. Thus, for example, the rankings and/or ratings may be divided into categories such as billing practices, cancellation practices, empathy, humor, response quickness, and/or other categories.

In certain such embodiments, practitioners may include one or more groups of practitioners (e.g., medical group, specialty, and/or other such categories). The ratings/rankings of a practitioner may, thus, be relative to other practitioners within the group, relative to other practitioners outside the group, and/or via other comparisons. Furthermore, in certain embodiments, the ratings/rankings may be compared between different groups. Thus, for example, the ratings/rankings may allow for sorting between the different practice groups or for sorting between all available practitioners. The ratings/rankings and may also allow for sorting between practitioners of a specific practice group.

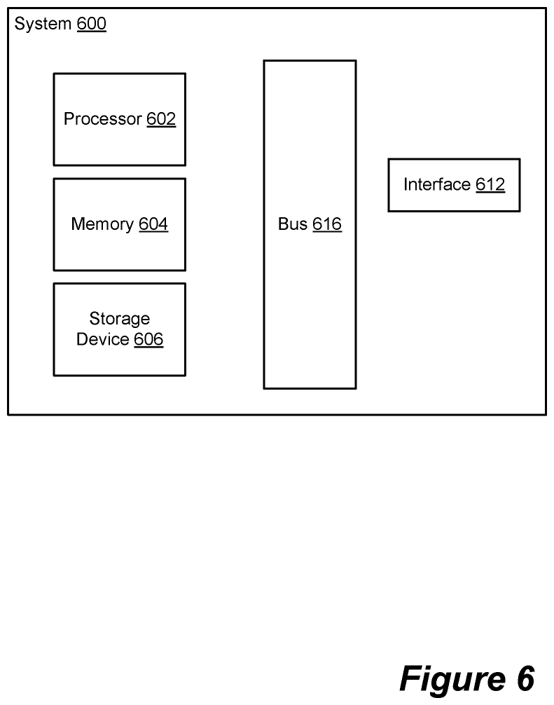

FIG. 6 illustrates a block diagram of a computing system, in accordance with one or more embodiments. According to various embodiments, a system 600 suitable for implementing embodiments described herein includes a processor 602, a memory module 604, a storage device 606, an interface 612, and a bus 616 (e.g., a PCI bus or other interconnection fabric.) System 600 may operate as variety of devices such as a server system such as an application server and a database server, a client system such as a laptop, desktop, smartphone, tablet, wearable device, set top box, etc., or any other device or service described herein.

Although a particular configuration is described, a variety of alternative configurations are possible. The processor 602 may perform operations such as those described herein. Instructions for performing such operations may be embodied in the memory 604, on one or more non-transitory computer readable media, or on some other storage device. Various specially configured devices can also be used in place of or in addition to the processor 602. The interface 612 may be configured to send and receive data packets over a network. Examples of supported interfaces include, but are not limited to: Ethernet, fast Ethernet, Gigabit Ethernet, frame relay, cable, digital subscriber line (DSL), token ring, Asynchronous Transfer Mode (ATM), High-Speed Serial Interface (HSSI), and Fiber Distributed Data Interface (FDDI). These interfaces may include ports appropriate for communication with the appropriate media. They may also include an independent processor and/or volatile RAM. A computer system or computing device may include or communicate with a monitor, printer, or other suitable display for providing any of the results mentioned herein to a user.

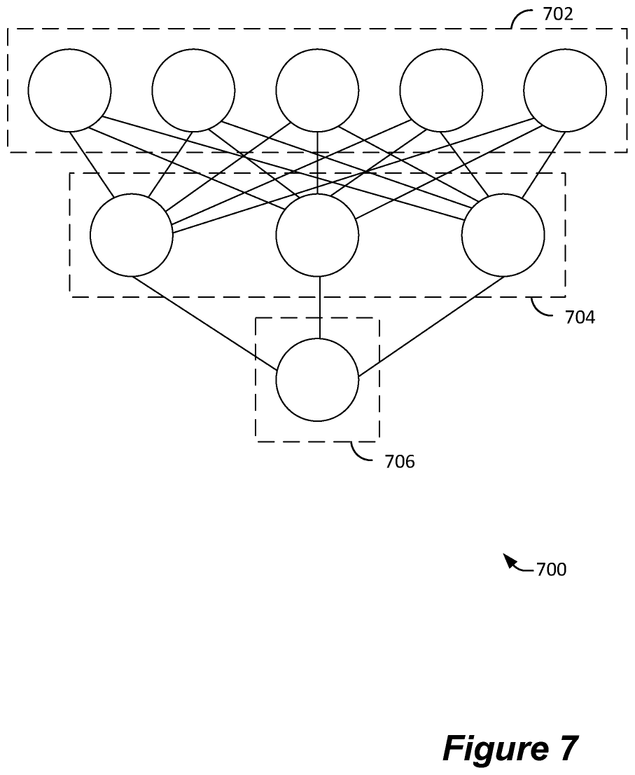

FIG. 7 illustrates a neural network, in accordance with one or more embodiments. FIG. 7 illustrates a neural network 700 that includes input layer 702, hidden layers 704, and output layer 706. Neural network 700 may be a machine learning network that may be trained to perform the techniques described herein.

Neural network 700 may be trained with inputs. Input layer 702 may include such inputs, which may be the training data described herein. Hidden layers 704 may be one or more intermediate layers where logic is performed to determine various aspects of the data (e.g., the category of practice of a response to a question of a survey, whether the response is an alphanumerical response, whether there is identifying data, and/or another such aspect). Output layer 706 may result from computation performed within hidden layers 704 and may output, for example, a practice rating, a point of improvement, feedback summary, and/or other such output.

Any of the disclosed embodiments may be embodied in various types of hardware, software, firmware, computer readable media, and combinations thereof. For example, some techniques disclosed herein may be implemented, at least in part, by non-transitory computer-readable media that include program instructions, state information, etc., for configuring a computing system to perform various services and operations described herein. Examples of program instructions include both machine code, such as produced by a compiler, and higher-level code that may be executed via an interpreter. Instructions may be embodied in any suitable language such as, for example, Java, Python, C++, C, HTML, any other markup language, JavaScript, ActiveX, VBScript, or Perl. Examples of non-transitory computer-readable media include, but are not limited to: magnetic media such as hard disks and magnetic tape; optical media such as flash memory, compact disk (CD) or digital versatile disk (DVD); magneto-optical media; and other hardware devices such as read-only memory (“ROM”) devices and random-access memory (“RAM”) devices. A non-transitory computer-readable medium may be any combination of such storage devices.

In the foregoing specification, various techniques and mechanisms may have been described in singular form for clarity. However, it should be noted that some embodiments include multiple iterations of a technique or multiple instantiations of a mechanism unless otherwise noted. For example, a system uses a processor in a variety of contexts but can use multiple processors while remaining within the scope of the present disclosure unless otherwise noted. Similarly, various techniques and mechanisms may have been described as including a connection between two entities. However, a connection does not necessarily mean a direct, unimpeded connection, as a variety of other entities (e.g., bridges, controllers, gateways, etc.) may reside between the two entities.

In the foregoing specification, reference was made in detail to specific embodiments including one or more of the best modes contemplated by the inventors. While various embodiments have been described herein, it should be understood that they have been presented by way of example only, and not limitation. For example, some techniques and mechanisms are described herein in the context of fulfillment. However, the disclosed techniques apply to a wide variety of circumstances. Particular embodiments may be implemented without some or all of the specific details described herein. In other instances, well known process operations have not been described in detail in order not to unnecessarily obscure the techniques disclosed herein. Accordingly, the breadth and scope of the present application should not be limited by any of the embodiments described herein, but should be defined only in accordance with the claims and their equivalents.

What is claimed is:

1. A method comprising:
receiving training data, wherein the training data is associated with post-treatment satisfaction surveys;
classifying portions of the training data as, at least, identifying sections or non-identifying sections;
training, with the identifying sections, a first machine learning model to determine identifying data of surveys;
training, with the non-identifying sections, a second machine learning model to determine an improvement point;
receiving a first post-treatment satisfaction survey associated with a patient of a provider;
determining, with the first machine learning model, one or more identifying data sections of the first post-treatment satisfaction survey;
anonymizing the identifying data sections of the first post-treatment satisfaction survey to create an anonymized first post-treatment satisfaction survey;
analyzing the anonymized first post-treatment satisfaction survey to determine an improvement point for the provider; and
outputting the improvement point to a user device of the provider.

2. The method of claim 1, further comprising:
determining that a treatment for the patient has finished;
determining that the patient is authorized to receive the first post-treatment satisfaction survey; and
providing, based on the determining that the treatment of the patient has finished and that the patient is authorized to receive the first post-treatment satisfaction survey, the first post-treatment satisfaction survey to an electronic device associated with the patient.

3. The method of claim 1, further comprising:
determining, based on the improvement point, a first course associated with the improvement point, wherein the outputting the improvement point comprises providing data associated with the first course.

4. The method of claim 1, further comprising:
identifying, from data within an electronic health record (EHR) database and based on the improvement point, a first service associated with the improvement point, wherein the outputting the improvement point comprises providing data associated with the first service.

5. The method of claim 1, wherein the determining the identifying data comprises determining a name, a date, and/or a description of treatment.

6. The method of claim 1, wherein the anonymizing the identifying data sections comprises converting textual responses of the first post-treatment satisfaction survey to a response category and a response rating.

7. The method of claim 1, further comprising:
determining a location of the patient, wherein the analyzing the anonymized first post-treatment satisfaction survey is based on the location, wherein the analyzing the anonymized first post-treatment satisfaction survey based on the location comprises determining the improvement point based on the location.

8. The method of claim 1, wherein the classifying portions of the training data further comprises classifying portions of testimonial data as numerical, essay, and/or testimonial data.

9. A system comprising:
a electronic health record (EHR) database;
a communications interface, communicatively coupled to the electronic health record (EHR) database via a network;
a processor, communicatively coupled to the communications interface and the EHR database and configured to cause the system to perform operations comprising:
receiving, with the communications interface, treatment process data from the EHR database;
determining, from the treatment process data, that a treatment for a first patient with a first provider has finished;
causing, based on the determining that the treatment for the first patient has finished, a first post-treatment satisfaction survey to be provided to an electronic device of the first patient;
receiving a first survey, wherein the first survey is a response to the first post-treatment satisfaction survey;
dividing the first survey into identifying sections and non-identifying sections;
removing the identifying sections;
determining, based on the non-identifying sections, a first improvement point for the first provider;
determining, from course data stored within the EHR database and based on the first improvement point, a first course associated with the first improvement point; and
transmitting first course data associated with the first course to a user device of the first provider.

10. The system of claim 9, wherein the operations further comprise:
receiving a training data;
classifying portions of the training survey as, at least, identifying sections or non-identifying sections;
training, with the identifying sections, a first machine learning model to determine identifying data of surveys; and
training, with the non-identifying sections, a second machine learning model to determine an improvement point for the provider.

11. The system of claim 10, wherein the dividing the first survey into the identifying sections and the non-identifying sections comprises determining, with the first machine learning model, one or more identifying data sections of the first survey, and wherein the removing the identifying sections comprises anonymizing the identifying data sections of the first survey to create an anonymized first survey.

12. The system of claim 11, wherein the determining the one or more identifying data comprises determining a name, a date, and/or a description of a treatment procedure within the first survey.

13. The system of claim 11, wherein the anonymizing the identifying data sections comprises converting textual responses of the first survey to a response category and a response rating.

14. The system of claim 11, wherein the determining the first improvement point comprises analyzing the anonymized first survey.

15. The system of claim 14, wherein the operations further comprise:
determining a location of the patient, wherein the analyzing the anonymized first survey is based on the location, wherein the first improvement point is determined based on the location.

16. The system of claim 9, wherein the dividing the first survey further comprises dividing into numerical, essay, and/or testimonial sections.

17. The system of claim 9, wherein the operations further comprise:
identifying, from service data stored within the DAR database and based on the first improvement point, a first service associated with the improvement point; and
transmitting first service data to the user device of the first provider.

18. A computer program product comprising computer-readable program code capable of being executed by one or more processors when retrieved from a non-transitory computer-readable medium, the program code comprising instructions configurable to cause operations comprising:
receiving a training data;
classifying portions of the training data as, at least, identifying sections or non-identifying sections;
training, with the identifying sections, a first machine learning model to determine identifying data of surveys;
training, with the non-identifying sections, a second machine learning model to determine an improvement point;
receiving a first post-treatment satisfaction survey associated with a patient of a provider;
determining, with the first machine learning model, one or more identifying data sections of the first post-treatment satisfaction survey;
anonymizing the identifying data sections of the first post-treatment satisfaction survey to create an anonymized first post-treatment satisfaction survey;
analyzing the anonymized first post-treatment satisfaction survey to determine an improvement point for the provider; and
outputting the improvement point to a user device of the provider.

19. The computer program product of claim 18, wherein the operations further comprise:
determining that a treatment for the patient has finished;
determining that the patient is authorized to receive the first post-treatment satisfaction survey; and
providing, based on the determining that the treatment of the patient has finished, the first post-treatment satisfaction survey to an electronic device associated with the patient.

20. The computer program product of claim 18, wherein the operations further comprise:
determining, based on the improvement point, a first course associated with the improvement point, wherein the outputting the improvement point comprises providing data associated with the first course.
